(19) United States
(12) Patent Application Publication
(10) Pub. No.: US 2023/0084202 A1
(43) Pub. Date: Mar. 16, 2023

(54) SECURE ARTIFICIAL INTELLIGENCE MODEL DEPLOYMENT AND INFERENCE DISTRIBUTION

(71) Applicant: GE Precision Healthcare LLC, Milwaukee, WI (US)

(72) Inventors: Abhijit Patil, Bengaluru (IN); Rakesh Mullick, Bangalore (IN); Bipul Das, Chennai (IN)

(21) Appl. No.: 17/474,711

(22) Filed: Sep. 14, 2021

(51) Int. Cl. G06F 21/62

(52) U.S. Cl. CPC G06F 21/6245

(57) ABSTRACT

Techniques are described that that facilitate securely deploying artificial intelligence (AI) models and distributing inferences generated therefrom. According to an embodiment, a system is provided that comprises a memory that stores computer executable components, and a processor that executes the computer executable components stored in the memory. The computer executable components comprise an algorithm execution component that applies an AI model to input data and generates output data, and an encryption component that encrypts the output data using a proprietary encryption mechanism, resulting in encrypted output data. The proprietary encryption mechanism can include a mechanism that prevents usage and rendering of the encrypted output data without decryption of the encrypted output data using a proprietary decryption mechanism.

### TECHNICAL FIELD

This application relates to computer-implemented techniques for securely deploying artificial intelligence (AI) models and distributing inferences generated therefrom.

### BACKGROUND

As trained artificial intelligence (AI) models are being deployed in larger numbers in the marketplace, the threat of competitors reverse engineering AI models is ever-present. This threat is particularly imminent in an environment in which AI models are distributed to customers in a networked or cloud-based architecture across multi-vendor sites. In such an environment, the possibility of leakage of original (unprocessed) and inferred (AI processed) data can be a conduit for competitors to accelerate their AI efforts and reverse-engineer the models while using existing AI applications as a predicate for fast regulatory approvals. Although there have been efforts directed toward encrypting and protecting AI models from being extricated and pirated, we currently do not have an approach instrumented to protect the reverse engineering of AI models based on leveraging the input data and the output data generated by the AI model as the ground truth using supervised learning methods to recreate alternate models with comparable performance.

### SUMMARY

The following presents a summary to provide a basic understanding of one or more embodiments of the invention. This summary is not intended to identify key or critical elements or delineate any scope of the different embodiments or any scope of the claims. Its sole purpose is to present concepts in a simplified form as a prelude to the more detailed description that is presented later. In one or more embodiments, systems, computer-implemented methods, apparatus and/or computer program products are described herein that facilitate securely deploying AI models and distributing inferences generated therefrom.

According to an embodiment, a system is provided that comprises a memory that stores computer executable components, and a processor that executes the computer executable components stored in the memory. The computer executable components comprise an algorithm execution component that applies an AI model to input data and generates output data, and an encryption component that encrypts the output data using a proprietary encryption mechanism, resulting in encrypted output data. The proprietary encryption mechanism can include a mechanism that prevents usage and rendering of the encrypted output data without decryption of the encrypted output data using a proprietary decryption mechanism.

The computer executable components can further comprise a decryption component that decrypts the encrypted output data using the proprietary decryption mechanism to obtain the output data, and a rendering component that renders the output data. In some implementations, the decryption component and the rendering component are included in a proprietary software application, and wherein the proprietary software application prevents exporting of the output data. With these implementations, the computer executable components can further comprise an orchestration component that provides the encrypted output data to the proprietary software application. For example, the proprietary software application can comprise a web-application accessible to external systems via one or more wireless communication networks. In some implementations, the orchestration component receives the input data from an external system in association with a request to process the input data via the AI model.

In another embodiment, a system is provided that comprises a memory that stores computer executable components, and a processor that executes the computer executable components stored in the memory. The computer executable components comprise a rendering application component that receives encrypted output data generated by an AI model. The rendering application component further includes a decryption component that decrypts the encrypted output data using a proprietary decryption mechanism, resulting in decrypted output data, and a rendering component that renders the decrypted output data via a suitable output device (e.g., a display, a speaker, etc.). The rendering application component can further include an export component that prevents exportation of the decrypted output data to unauthorized entities.

In some embodiments, elements described in connection with the disclosed systems can be embodied in different forms such as a computer-implemented method, a computer program product, or another form.

### DESCRIPTION OF THE DRAWINGS

FIG. 1 illustrates a block diagram of an example, non-limiting system that facilitates securely deploying AI models and distributing inferences generated therefrom, in accordance with one or more embodiments of the disclosed subject matter.

FIG. 2 illustrates another example system that facilitates securely deploying AI models and distributing inferences generated therefrom, in accordance with one or more embodiments of the disclosed subject matter.

FIG. 3 illustrates another example system that facilitates securely deploying AI models and distributing inferences generated therefrom, in accordance with one or more embodiments of the disclosed subject matter.

FIG. 4 illustrates a block diagram of an example, non-limiting computer implemented method that facilitates securely deploying AI models and distributing inferences generated therefrom, in accordance with one or more embodiments of the disclosed subject matter.

FIG. 5 illustrates a block diagram of another example, non-limiting computer implemented method that facilitates securely deploying AI models and distributing inferences generated therefrom, in accordance with one or more embodiments of the disclosed subject matter.

FIG. 6 illustrates a block diagram of another example, non-limiting computer implemented method that facilitates securely deploying AI models and distributing inferences generated therefrom, in accordance with one or more embodiments of the disclosed subject matter.

FIG. 7 illustrates a block diagram of an example, non-limiting operating environment in which one or more embodiments described herein can be facilitated.

FIG. 8 illustrates a block diagram of another example, non-limiting operating environment in which one or more embodiments described herein can be facilitated.

FIG. 9 illustrates a block diagram of another example, non-limiting operating environment in which one or more embodiments described herein can be facilitated.

FIG. 10 illustrates a block diagram of another example, non-limiting operating environment in which one or more embodiments described herein can be facilitated.

FIG. 11 illustrates a block diagram of another example, non-limiting operating environment in which one or more embodiments described herein can be facilitated.

FIG. 12 illustrates a block diagram of another example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Background section, Summary section or in the Detailed Description section.

The disclosed subject matter is directed to techniques that facilitate securely deploying AI models and distributing inferences generated therefrom. The main challenge the disclosed techniques proactively address is the prevention of unauthorized leakage of AI model processed output data, thereby preventing its usage by unauthorized entities to rapidly reverse engineer the AI models in a supervised learning framework using the output data as the ground truth. In this regard, if the receiver of any AI solution is able to access the input and output data (enabled typically when an optional AI service is invoked at will), the end user can potentially use that data to retrain a new deep-learning model/architecture and push the performance to match that of the original AI model. The disclosed techniques aim to restrict the export of the AI model output data while allowing the user to share the original input data, thereby preventing piracy of the AI model.

To facilitate this end, the disclosed techniques employ a distributed AI model deployment architecture wherein the proprietary AI models are executed by a centralized AI model orchestration service controlled by the AI model provider. The centralized AI model orchestration service further provides the AI model inference outputs to the customer in a controlled manner by introducing usage limitations on the AI model inference outputs provided thereto. In one or more embodiments, the AI model orchestration service encrypts or encodes the AI model output data in a manner that renders it exclusively capable of being rendered, opened, or otherwise interoperable on proprietary display/reporting sub-systems with the necessary decrypting/decoding ability of the embedded value-added information. In various embodiments, the proprietary display/reporting sub-systems can be or include a proprietary consumer application via which the end user can receive the AI model inference output in an encrypted/encoded form that the proprietary consumer application is configured to decrypt and render. The proprietary consumer application can further be configured to minimize or prohibit the unauthorized export of the AI model output data following decryption and rendering. This further creates the demand and adoption of modern services to visualize and report on AI solutions while preventing competitors from recreating equivalent models to those released as products and thereby preserve the differentiation of the service.

The disclosed techniques can be applied in various domains in which proprietary AI model inferences are provided to consumers, and more particularly to consumers with access to the input data. For instance, the disclosed techniques can be applied in the healthcare domain in association with providing clinicians with inference outputs generated by clinical inferencing models applied to their internal clinical input data. On example use case in this context can includes the usage of proprietary AI models adapted to generate inferences on consumer provided medical image data to facilitate clinical review and radiologist reporting. For example, the clinical inferencing models can include a range of AI models adapted can detect patterns, features and artifacts in signal and medical image data to perform diagnostic tasks, image enhancement tasks, organ segmentation tasks, and the like. With these embodiments, the algorithm orchestration service can employ a centralized, network accessible algorithm orchestration component that serves as middle layer connecting medical image providers with AI model processing web services for executing AI models on their medical images and returning consumable outcomes/results.

In accordance with this use case, the algorithm orchestration component can receive a medial image (or images) in association with a request to process the medical image using one or more proprietary AI image processing algorithms. In some embodiments, image providers can identify or indicate the specific AI algorithms to apply to a medical image in association with provision of the image processing request. Additionally, or alternatively, the algorithm orchestration component can determine what workflows and associated AI algorithms to apply to a given image based on metadata associated with the image and the information included in the algorithm catalog. With these embodiments, the algorithm orchestration component can automatically select at least one AI model to execute on a medical image. The AI models can include, but are not limited to, image restoration algorithms (e.g., used to improve the quality of the image), image analysis algorithms (e.g., classification/diagnosis models, organ segmentation models, etc.), image synthesis algorithms (e.g., used construct a three-dimensional image based on multiple two-dimensional images images), image enhancement algorithms (e.g., used improve the image by using filters or adding information that will assist with visualization), and image compression algorithms (e.g., used to reduce the size of the image to enhance transmission times and storage required). In various embodiments, the algorithm execution process involves employing an algorithm execution component to execute the one or more proprietary AI algorithms by calling/invoking the algorithms at their network accessible file source (e.g., using their corresponding application program interface (API) calls as defined by the workflow code/logic).

The algorithm orchestration component can further provide the requesting entity with the AI model results/outcomes in an encrypted/encoded format via a proprietary rendering application that the proprietary rendering application is configured to decode/decrypt. For example, the proprietary rendering application can include a medical imaging visualization application that provides for rendering AI generated enhanced medical images, organ segmentation masks, diagnostic results and so one generated by one or more clinical AI models. The proprietary rendering application can also include tools that facilitate calling and directing the AI orchestration service to execute the desired AI models and return the encrypted results thereto. The proprietary rendering application can further be configured to prevent or restrict the export of the decoded/decrypted clinical AI model inference output data.

Although various embodiments of the disclosed systems are described in association with securely deploying medical image AI models and distributing inference outputs generated therefrom, it should be appreciated that the disclosed systems can be tailored to facilitate clinical decision support in many other clinical and operational healthcare domains to eliminate human error and inefficiency, improve patient quality of care and reduce waste, etc. In this regard, medical imaging is just one out of a thousand use cases in which the disclosed techniques for integrating AI informatics to facilitate making healthcare related decisions and evaluations can be applied. The disclosed distributed AI model deployment architecture can be used to securely distribute clinical inferencing model inference outputs in association with their integration into clinical workflows of various healthcare disciplines in a manner that can at scale across departments, enterprises and regions. For example, in addition to facilitating clinical workflow optimization for radiologists, physicians and other clinicians involved with patient care, the disclosed techniques can provide for integrating AI informatics at the administrative level to facilitate planning, regulating, and managing medical services. For example, AI informatics can be integrated into patient scheduling and bed management systems to optimize population heath management. In another example, AI informatics can be used to identify areas of a healthcare system associated with waste and facilitate determining techniques for reducing costs and improving return on investment (ROI), or otherwise optimizing clinical and financial outcomes of patient care delivery. AI informatics can be integrated into healthcare billing systems to facilitate improving claim submission and reimbursement efficiency, to facilitate reducing fraud waste and abuse (FWA), and the like.

The disclosed techniques can also be extended to various other industries or domains in addition to healthcare. For example, the disclosed distributed learning techniques can be extended to the marketing industry to automatically identify trends and deliver more personalized advertisements and products to consumers. In another example, the disclosed distributed learning techniques can be applied in the transportation industry to facilitate autonomous driving systems, to optimize airline and train scheduling and ROI in real-time based on anticipated delays, and the like. In another example, the disclosed distributed learning systems can provide various AI solutions to business organization to sift through huge data pools, process applications, spot anomalies, draw conclusions and make informed decisions, etc., to facilitate increasing service quality and efficiency while reducing cost. Other industries that can employ the disclosed distributed learning architecture to facilitate integrating AI informatics into their systems can include for example, educational systems, manufacturing systems, legal systems, personalized assistance systems, government regulatory systems, security systems, machine-to-machine (M2M) communication systems, agriculture systems, etc. The possibilities are endless.

The terms “algorithm” and “model” are used herein interchangeably unless context warrants particular distinction amongst the terms. The types of AI and ML models or algorithms that the disclosed techniques are designed to securely deploy and protect can vary. In this regard, the particular inferencing task and/or model architecture can vary. The term “clinical inferencing model” is used herein to refer to a AI/ML model configured to perform a clinical decision/processing on clinical data. The clinical decision/processing task can vary. For example, the clinical decision/processing tasks can include classification tasks (e.g., disease classification/diagnosis), disease progression/quantification tasks, organ segmentation tasks, anomaly detection tasks, image reconstruction tasks, and so on. The clinical inferencing models can employ various types of ML algorithms, including (but not limited to): deep learning models, neural network models, deep neural network models (DNNs), convolutional neural network models (CNNs), generative adversarial neural network models (GANs), long short-term memory models (LSTMs), attention-based models, transformers and the like.

As used herein, a “medical imaging inferencing model” refers to an image inferencing model that is tailored to perform an image processing/analysis task on one or more medical images. For example, the medical imaging processing/analysis task can include (but is not limited to): image reconstruction, image enhancement, scan series characteristic classification, disease/condition classification, disease region segmentation, organ segmentation, disease quantification, disease/condition staging, risk prediction, temporal analysis, anomaly detection, anatomical feature characterization, medical image reconstruction, and the like. The terms “medical image inferencing model,” “medical image processing model,” “medical image analysis model,” and the like are used herein interchangeably unless context warrants particular distinction amongst the terms.

The term “image-based inference output” is used herein to refer to the determination or prediction that an image processing model is configured to generate. For example, the image-based inference output can include a segmentation mask, a reconstructed image, an enhanced image, an adapted image, an annotated image, a classification, a value, or the like. The image-based inference output will vary based on the type of the model and the particular task that the model is configured to perform. The image-based inference output can include a data object that can be rendered (e.g., a visual data object), stored, used as input for another processing task, or the like. The terms “image-based inference output”, “inference output” “inference outcome,” “inference result” “inference”, “output”, “outcome,” “predication”, and the like, are used herein interchangeably unless context warrants particular distinction amongst the terms. The outputs can be in different formats, such as for example: a Digital Imaging and Communications in Medicine (DICOM) structured report (SR), a DICOM secondary capture, a DICOM parametric map, an image, text, and/or JavaScript Object Notation (JSON).

The types of medical images processed/analyzed by the medical image inferencing models described herein can include images captured using various types of image capture modalities. For example, the medical images can include (but are not limited to): radiation therapy (RT) images, X-ray (XR) images, digital radiography (DX) X-ray images, X-ray angiography (XA) images, panoramic X-ray (PX) images, computerized tomography (CT) images, mammography (MG) images (including a tomosynthesis device), a magnetic resonance imaging (MRI) images, ultrasound (US) images, color flow doppler (CD) images, position emission tomography (PET) images, single-photon emissions computed tomography (SPECT) images, nuclear medicine (NM) images, and the like. The medical images can also include synthetic versions of native medical images such as synthetic X-ray (SXR) images, modified or enhanced versions of native medical images, augmented versions of native medical images, and the like generated using one or more image processing techniques. The medical imaging processing models disclosed herein can also be configured to process 3D images.

The term “web platform” as used herein refers to any platform that enables delivery of content and services over a network (i.e., the web/Internet) using a network transfer protocol, such as HTTP, sFTP, or another network transfer protocol. For example, a web platform can include, but is not limited to, a web-application (i.e., an interactive website), a mobile website, a mobile application or the like. The terms “web platform,” “web-based platform,” “network platform,” “platform,” and the like are used herein. interchangeably unless context warrants particular distinction amongst the terms.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

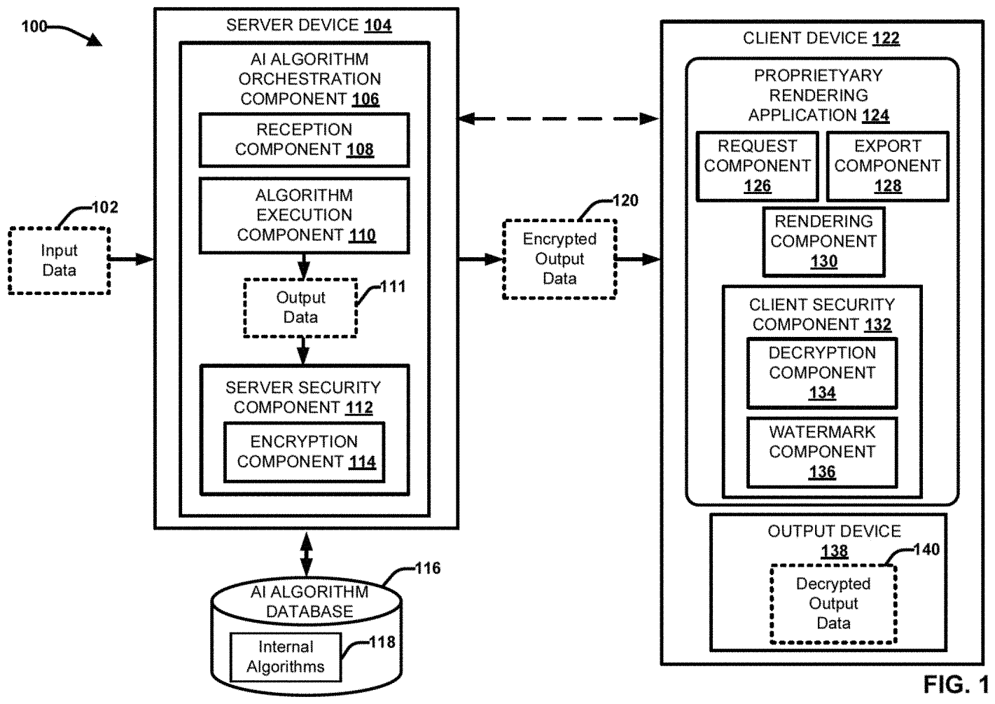

FIG. 1 illustrates a block diagram of an example system 100 that facilitates securely deploying AI models and distributing inferences generated therefrom in accordance with one or more embodiments of the disclosed subject matter. Embodiments of systems described herein can include one or more machine-executable components embodied within one or more machines (e.g., embodied in one or more computer-readable storage media associated with one or more machines). Such components, when executed by the one or more machines (e.g., processors, computers, computing devices, virtual machines, etc.) can cause the one or more machines to perform the operations described.

In this regard system 100 includes an AI algorithm orchestration component 106 that facilitates securely deploying proprietary AI models to input data 102 and distributing inference output data 111 generated therefrom in an encrypted/encoded form, represented in FIG. 1 as encrypted output data 120. System 100 further includes a proprietary rendering application 124 that provides for receiving the encrypted output data 120 and decrypting the encrypted output data 120 to facilitate rendering and using the decrypted output data 140. The AI algorithm orchestration component 106 and the proprietary rendering application 124 can respectively be and include machine-executable components embodied within one or more machines (e.g., embodied in one or more computer-readable storage media associated with one or more machines), which when executed by the one or more machines (e.g., processors, computers, computing devices, virtual machines, etc.) can cause the one or more machines to perform the operations described.

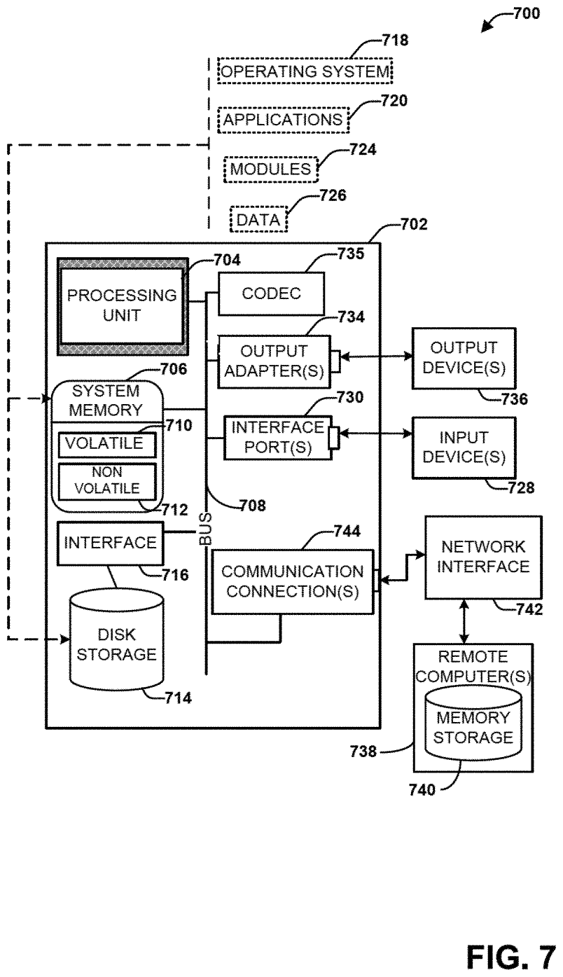

For example, in the embodiment shown, the AI algorithm orchestration component 106 is associated with a server device 104 and the proprietary rendering application 124 is associated with a client device 122. The server device 104 be or correspond to one or more real or virtual (e.g., cloud-based) computing devices that include or are operatively coupled to memory (not shown) that stores the AI algorithm orchestration component 106 and/or one or more of the components associated therewith (e.g., the reception component 108, the algorithm execution component 110, the server security component 112 and the encryption component 114). The server device 104 can further include or be operatively coupled to at least one processor (or processing unit) that executes the AI algorithm orchestration component 106 and/or one or more of the components associated therewith. Examples of said and memory and processor as well as other suitable computer or computing-based elements, can be found with reference to FIG. 7 (e.g., with reference to processing unit 704 and system memory 706), and can be used in connection with implementing one or more of the systems or components shown and described in connection with FIG. 1 or other figures disclosed herein.

Likewise, the client device 104 can be or correspond to one or more real or virtual (e.g., cloud-based) computing devices that include or are operatively coupled to memory (not shown) that stores the proprietary rendering application 124 and/or one or more of the components associated therewith (e.g., the request component 126, the export component 128, the rendering component 130, the client security component 132, the decryption component 134 and the watermark component 136). The server device 104 can further include or be operatively coupled to at least one processor (or processing unit) that executes the proprietary rendering application 124 and/or one or more of the components associated therewith. Examples of said and memory and processor associated with the client device 122 as well as other suitable computer or computing-based elements, can also be found with reference to FIG. 7 (e.g., with reference to processing unit 704 and system memory 706), and can be used in connection with implementing one or more of the systems or components shown and described in connection with FIG. 1 or other figures disclosed herein.

In the embodiment shown, the proprietary rendering application 124 is associated with a client device 122 to indicate that client device 122 can access and employ the AI orchestration services provided by the AI algorithm orchestration component 106 in a server client relationship. In this regard, the server device 104 and the client device 122 can be communicatively coupled to one another via one or more wired or wireless communication networks (represented by the dashed arrow line connecting the respective devices in system 100). For example, the proprietary rendering application 124 can include a web-application, a thin-client application, a hybrid application, a mobile application, or the like, via which the client device can access and employ the AI algorithm orchestration services provided by the AI algorithm orchestration component 106 via one or more wired or wireless communication networks. For instance, using the proprietary rendering application, the client device 122 can receive encrypted output data 120 generated by the AI algorithm orchestration component 106 and decrypt the encrypted output data 120 to generate decrypted output data 140 that can be rendered at the client device using a suitable output device 138. The type of the output device 138 can vary depending on the type of the decrypted output data 140. For example, the output device 138 can include a display device that renders the decrypted output data 140 in a visible format, a speaker, or the like. However, the architecture of system 100 can vary and is not limited to the server-client relationship presented. For example, in other embodiments, the proprietary rendering application 124 can include a native or local client application.

System 100 further includes an AI algorithm database 116 that includes one or more internal algorithms 118. The internal algorithms can include proprietary AI algorithms or models that can be accessed by the AI algorithm orchestration component 106 and executed by the algorithm execution component 110 on corresponding input data 102 to generate inference results, represented in FIG. 1 as output data 111. The AI algorithm orchestration component 106 can further provide the inference results to the client device 122 for rendering via the proprietary rendering application 124. However, in association with providing the inference results to the client device 122, the server security component 112 can employ one or more mechanisms to ensure the inference output data is provided to the client device 122 in a secure manner that prevents or minimizes usage thereof to reverse engineer the AI model that generated the result.

For example, the sever security component 112 can include an encryption component 114 that encrypts the AI model inference output data 111 using one or more proprietary encryption or encoding mechanisms to generate encrypted output data 120. In this regard, the encrypted output data 120 corresponds to the AI model inference output data 111 in an encrypted or encoded format. The proprietary rendering application 124 can further include a client security component 132 that facilitates ensuring the security of the AI model inference output data in association with provision to the client device 122. In various embodiments, the client security component 132 includes a decryption component 134 that decrypts the encrypted output data 120 using a proprietary decryption or decoding mechanism to generate the decrypted output data 140. The decrypted output data 140 can be or correspond to the AI model inference output data 111 prior to encryption/encoding by the encryption component 114. The proprietary rendering application 124 can further include rendering component 130 that renders the decrypted output data 140 via the output device 138. In this regard, the encrypted output data 120 can be encrypted or encoded in a manner that prevents the rendering component 130 from rendering it unless it is decrypted/decoded by the decryption component 134 using the proprietary decryption mechanism.

The proprietary encryption/encoding and decryption/decoding mechanism employed by the server security component 112 and the client security component 132 respectively can vary. In this regard, the encryption as encryption/encoding and decryption/decoding mechanism employed by the server security component 112 and the client security component 132 can include any mechanism for the conversion of the output data 111 from a readable format into an encoded format that can only be read or processed by the rendering component 130 after it's been decrypted. by the decryption component 134. In some embodiments, the proprietary encryption/encoding mechanism can include embedding one or more private tags on or within the output data 111 to generate the encrypted output data 120.

For example, in some implementations described in greater detail below, the output data 111 can include a medical image and/or medical image data formatted according to an interchangeable standard format (e.g., DICOM or the like). With these embodiments, the encryption component 114 can embed one or more private tags on or within the medical image/medial image data according to the interchangeable standard format. The decryption component 134 can further be configured to decode the encrypted output data 120 embedded in the private tags to render the full-fidelity medical image data. In some implementations of these embodiments, the input data 102 can include a medical image and the output data 111 can include a modified version of the medical image. For example, the AI algorithm applied to the input medical image can include an AI algorithm that changes the input medical image by enhancing the quality of the input image (e.g., via removing artifacts, enhancing the resolution, performing image registration, performing image harmonization, etc.), generating a reconstructed version of the input image from a different perspective, applies a segmentation mask to the input image, marking anatomical landmarks, and so on. With these implementations, the encryption component 114 may encrypt the entirety of the output data 111 or only the portion of the output data that differs from the input data 102 with private tags. For instance, the encryption component 114 can embed private tags on or within the output image that encodes only the image changes generated by the AI algorithm applied to the input image (e.g., the differences between the input image and the output image). In accordance with these embodiments, the decryption component 134 can be configured to identify and read the private tags to generate/extract the full-fidelity output image. However, non-proprietary rendering application without the decryption component will be unable to identify and read the private tags to generate/extract the full-fidelity output image. Still in other embodiments applied to image output data 111, the encryption component 114 can be configured to scramble the image data in a way that the fidelity of the image data required for the intended usage of the AI model processed image data is not impacted (e.g., performing clinical analysis review), but prevents or minimizes the ability to use the scrambled image data as ground truth data in association with attempting to reverse engineer the AI model (e.g., preventing reverse engineering of the quality enhance image data features).

Additionally, or alternatively, the proprietary encryption/encoding and decryption/decoding mechanism can include using paired encryption and decryption keys in accordance with systems and/or asymmetric key systems. With these embodiments, the encryption component can encrypt the output data 111 using a proprietary encryption key and the decryption component 134 can decrypt the encrypted output data 120 using a corresponding proprietary decryption key. Some suitable encryption algorithms that can be used by the encryption component 114 to encrypt the output data 111 can include but are not limited to: the advanced encryption standard (AES) algorithm, triple data encryption standard algorithms triple DES), the Rivest-Shamir-Adleman (RSA) encryption algorithm, the blowfish encryption algorithm, and the two fish encryption algorithm.

The proprietary rendering application 124 can further include one or more mechanisms that prevent or minimize the exportation of the decrypted output data 140 in association with rendering of the decrypted output data 140. In some embodiments, the proprietary rendering application 124 can include an export component 128 that controls exportation of the decrypted output data 140. In some implementations, the export component 128 can prevent the exportation of the decrypted output data to any system or device in association with usage of the proprietary rendering application 124. For example, the export component 128 can prevent downloading the decrypted output data 140, saving the decrypted output data (e.g., to local memory of the client device 122 and/or a removable memory storage device), and/or sending the decrypted output data 140 to another system or device. In other implementations, the export component 128 can prevent the exportation of the decrypted output data to any system to defined unauthorized entities (e.g., black-listed systems, devices, network addresses, etc.,). The client security component 132 can also include a watermark component 136 that can embed a proprietary digital watermark on or withing the decrypted output data 140 in association with decryption and rendering and/or in response to initiation of an exportation action (e.g., saving, storing, sending, downloading, attaching to a message, etc.). The proprietary digital watermark can scramble the decrypted output data or modify the decrypted output data in a way that the fidelity of the decrypted output data 140 required for the intended usage of thereof is not impacted (e.g., performing clinical analysis review), but prevents or minimizes the ability to use the scrambled or modified decrypted output data 140 data as ground truth data in association with attempting to reverse engineer the AI model (e.g., preventing reverse engineering of the quality enhance image data features).

The type of the internal algorithms 118, the type of the input data 102, the source of the input data 102, the type of the output data 111, and the process for effectuating application of one or more internal algorithms 116 to the input data 102 and providing the encrypted output data 120 to the client device 122/proprietary rendering application 124 can vary. For example, the input data 102 and/or the output data 111 can include various data formats and data types, including image data, metadata, text data, audio data, and other forms of electronic signal data. In some embodiments, the input data 102 can be received from the client device 122 and/or the proprietary rendering application 124. In other embodiments, the input data 102 can be received from one or more proprietary data sources associated with the server device 104 and/or the AI algorithm database 116. Still in other embodiments, the input data 102 can be received from one or more non-proprietary (or external) data sources associated with the client device 122 and/or end-consumer/customer. Regardless of the source of the input data 102, the reception component 108 can employ suitable mechanism to identify, extract and or otherwise receive the input data 102 for processing by the algorithm execution component 110 and the server security component 112. In some embodiments in which the input data 102 is not received directly from the end-consumer/customer in association with instructions to process the input data 102 using one or more of the internal algorithms 116, the AI algorithm orchestration component 106 can also be configured to provide the end-consumer/customer with the (unprocessed) input data 102 in addition to the encrypted output data 120. For example, the AI algorithm orchestration component 106 can provided the client device 122 and/or the proprietary rendering application 124 with the (unprocessed) input data and the encrypted output data 120.

In some embodiments, the proprietary rendering application 124 can include a request component 126 that provides for requesting application of one or more of the internal algorithms 118 to input data 102. For example, the request component 126 can provide an AI algorithm processing request function of the proprietary rendering application 124 via which a user can provide input identifying or indicating one or more of the internal algorithms 118 desired for running on a particular input data object and/or input data set. For instance, as applied to a medical imaging application that provides for displaying and reviewing medial image data, the request component 126 can provide for requesting application of one or more AI medical image processing algorithms to medical image data access and viewed via the medical imaging application. For example, in association with using the medical imaging application, a user can provide input requesting application of an image enhancement model, an image segmentation model, an image restoration model, an image reconstruction model, and so on, to a particular medical image and/or medical image study. With these embodiments, the request component 126 can provide AI model processing request to the AI algorithm orchestration component 106 identifying or indicating a desired internal algorithm for application to the input data object or input data set. The reception component 108 can further receive the AI model processing requests. In some implementations, the client device 122 can also provide the input data 102 with the AI model processing request, which can also be received by the reception component 108. Additionally, or alternatively, the AI model processing request can identify the desired input data 102 for processing by the one or more AI models and include information that enables the reception component 108 to access and retrieve the corresponding input data 106 at its network accessible location.

In one or more exemplary embodiments, system 100 can be adapted to facilitate the secure usage of proprietary clinical inferencing models by third-party consumers in the healthcare industry, such as medical image providers. In this regard, system 100 can be adapted to orchestrate application of medical imaging AI models on medical images provided by the medical image providers, wherein the medial image AI models are accessed by the consuming entity via the proprietary rendering application 124 as web-services and/or wrapped jobs. The medical image providers can include essentially any entity that provides medical images as the input data 102 for processing by the AI algorithm orchestration component 106. For example, a medical image provider can include a healthcare system, a hospital system, a medical imaging system, an individual clinical/radiologist (e.g., a sole practitioner) or the like. The medical image provider can include a client entity that uses the client device 122 and/or the proprietary rendering application 124, and or another entity. In some embodiments, the server device 104 can access and/or receive the input medial image data (e.g., the input data 102) provided by the medial image providers in one or more input data sources/systems that are communicatively coupled to the server device 104. For example, the one or more input data sources/systems can include one or more data sources that store medical images and/or information related to the medical images, such as metadata describing various attributes of the medical images, radiology reports associated with the medical images, relevant patient information, and the like. For example, the attributes can refer to patient data associated with the medical image such as name, age, previous medical history data, and/or other data relating to the patient and the medical image. Attributes can also describe exam series and image level metadata to identify and/or execute related workflows. For example, the metadata associated with the medical images can include information regarding (but not limited to), image acquisition protocols, image quality, signal noise, image index size, matrix size, resolution, orientation, capture modality, type and dimensions of anatomical features depicted in the image data, image rating, image quality, image storage location (or locations), image capture time, and other features. The metadata tags can also include (but are not limited to) information describing the image type, the anatomical part depicted in the image data, the medical condition/pathology reflected in the image data, patient demographics, and relevant patient medical history factors.

In this regard, the source of the input data 102 can comprise different types of data sources that are provided by the same entity/organization that owns/operates the algorithm orchestration component 104 as well as provided by various different third party or external entities/organizations. For example, the source of the input data 102 can include or be communicatively coupled to one or more internal medical image databases, one or more external medical image databases, one or more internal and external workstations, one or more internal and external picture archiving communication systems (PACS) and consoles, and so forth. The source of the input data 102 can be located on the same network as well as across multiple networks and regions around the world. The number of image provider systems/devices that can provide the input data 102 is unlimited.

In this regard, the term “internal” as used herein to refers to a proprietary data source/system associated with the enterprise that owns, provides, manages and/or controls the internal algorithms 118 and the features and functionalities of the AI algorithm orchestration component 106. For example, in some implementations, the single enterprise can be a health information technology (HIT) company (such as General Electric (GE) Healthcare Corporation) that provides a range of products and services that include medical imaging and information technologies, electronic medical records, medical diagnostics, patient monitoring systems, drug discovery, biopharmaceutical manufacturing technologies and the like. According to this example, the HIT company can also provide, manage, and/or control the algorithm orchestration component 106 as well as employ the algorithm orchestration component 106 to process their own medical images using their internal algorithms 118. The term “external” as used herein to refer a system, device and/or medical image data source that is owned and/or operated by a third-party entity that does not provide, manage, and/or control the algorithm orchestration component 106 and the internal algorithms.

In accordance with these embodiments, the AI algorithm orchestration component 106 can receive image processing requests 106 from third-party medical image providers that correspond to requests to apply one or more internal algorithms 118 to a medical image or group of medical images, such as a group of medical images included in particular imaging study. These image processing algorithms can include various medical image inferencing algorithms or models (e.g., AI models). For example, the image processing algorithms can include, but are not limited to, image restoration algorithms (e.g., used to improve the quality of the image), image analysis algorithms (e.g., classification/diagnosis models, organ segmentation models, etc.), image synthesis algorithms (e.g., used construct a three-dimensional image based on multiple two-dimensional images images), image enhancement algorithms (e.g., used improve the image by using filters or adding information that will assist with visualization), and image compression algorithms (e.g., used to reduce the size of the image to enhance transmission times and storage required).

In the embodiment shown, the internal algorithms 118 are stored at an AI algorithm database 118 that can be accessed and applied by the algorithm execution component 110 to the input data 102. In various embodiments, the internal algorithms 118 algorithms can be integrated into the predefined workflows as HTTP tasks and accessed and applied to the medical images as web-services in accordance with the pre-defined workflows. Additionally, or alternatively, the algorithms/models can be integrated into workflows as “jobs.” Specifically, an algorithm/model and other tasks defined by computer executable instructions can be wrapped in a Kubernetes Job function and the algorithm orchestration component can execute it asynchronously inside the cluster on behalf of the user. This feature opens multiple possibilities specially related to legacy systems where the client service is not under HTTP and is only a simple command line and/or executable. In this regard, system 100 can enable various different medical image providers to access and employ the algorithm orchestration component 106 to process their medical images using various proprietary medical image inferencing algorithms and obtain the results in a secure environment that prevents or minimizes usage of the results to pirate the proprietary algorithms.

While one or more elements of system 100 are illustrated as separate components, devices and/or data structures, it is noted that the elements can be comprised of one or more other elements. In this regard, the architecture of system 100 can vary. In addition, although a single client device 122 and rendering application 124 are depicted, it should be appreciated that the number of client devices and/or rendering applications connected to the server device 104 and/or the AI algorithm orchestration component 106 is unlimited. Further, it is noted that the embodiments can comprise additional components not shown for sake of brevity. Additionally, various aspects described herein may be performed by one device or two or more devices in communication with each other. For example, one or more systems, services, devices, applications, components and the like, of system 100 can be located on separate (real or virtual) machines and accessed via a network (e.g., the Internet).

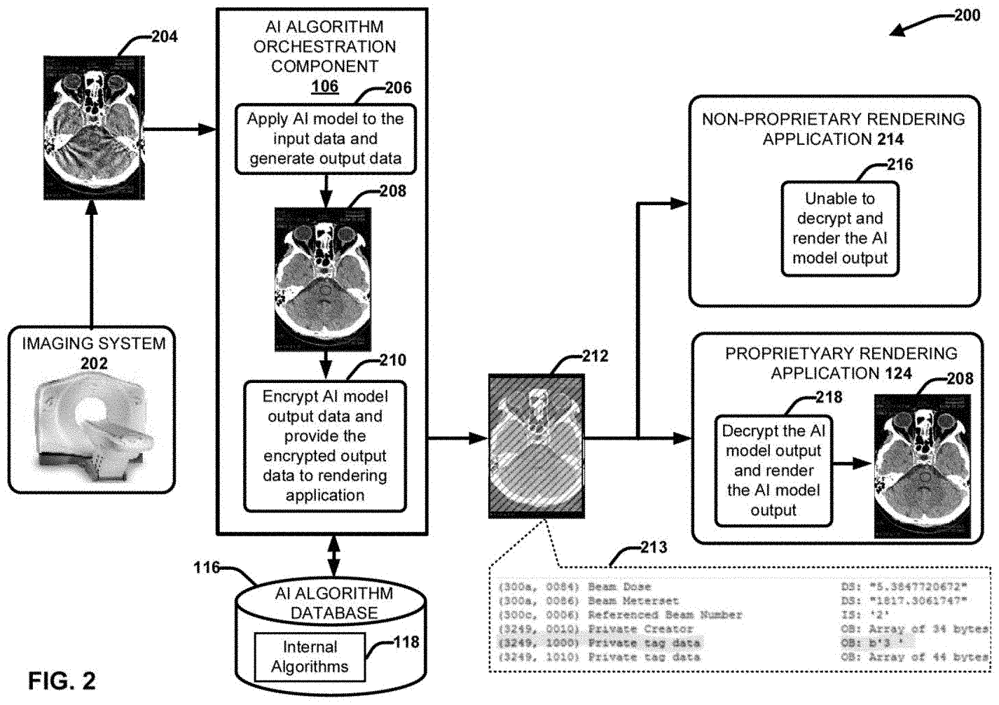

FIG. 2 illustrates another example system that facilitates securely deploying AI models and distributing inferences generated therefrom, in accordance with one or more embodiments of the disclosed subject matter. System 200 particularly demonstrates an implementation of system 100 as applied in association with processing medical images using one or more medical image inferencing models and securely distributing the output data. In this example, the input data include a medical image 204 and the output data 208 includes a modified version of the medical image. For example, in some implementations, the modified version of the medical image can include an enhanced version of the medical image 204 generated by one or more medical image enhancement AI models (e.g., resolution enhancement models, artifact removal models, image harmonization models, image reconstruction models, etc.). It should be appreciated that the type of image processing model applied to the input medical image 204 to generate the output data can vary.

System 200 can include same or similar components, devices and elements as system 100. Repetitive description of like elements employed in respective embodiments is omitted for sake of brevity. For example, it should be appreciated that certain components associated with the AI algorithm orchestration component 106 and the proprietary rendering application 124 described with reference to FIG. 1 are removed in FIG. 2 for ease/simplicity of illustration.

In accordance with system, 200, the input image medical image 204 can be received by the AI algorithm orchestration component 106 directly from an imaging system 202 used to capture/generate the medical image. However, the source of the input image can vary. System 200 further demonstrates a high-level methodology for processing the input image by the AI algorithm orchestration component 106 and distributing the output data 208. In this regard, at 206, the AI algorithm orchestration component 106 applies an AI model to the input data and generates the output data 208 (e.g., using algorithm execution component 110). The AI model applied can include one or more internal algorithms 116 accessed by the model execution component 110 in the AI algorithm database 118. At 210, the AI algorithm orchestration component 106 further encrypts the AI model output data to generate encrypted output data 212 and provides the encrypted output data to one or more rendering applications. In this example, the encrypted output data 212 is encrypted using one or more private tags in accordance with a standard interactable data format (e.g., DICOM, HL7, or the like), as indicated by call out box 213. System 200 further demonstrates the utility of the proprietary rendering application 124 relative to a non-proprietary rendering application that does not include the client security component 132. In this regard, when the encrypted output data is provided to the non-proprietary rending application, at 216, the application is unable to decrypt and render the AI model output. However, based on provision of the encrypted output data 212 to the proprietary rendering application, at 218, the proprietary rendering application decrypts the AI model output and renders the AI model output in a decrypted form (e.g., using decryption component 134 and rendering component 128), which in this example includes the original AI model output data 208, the modified version of the input medical image.

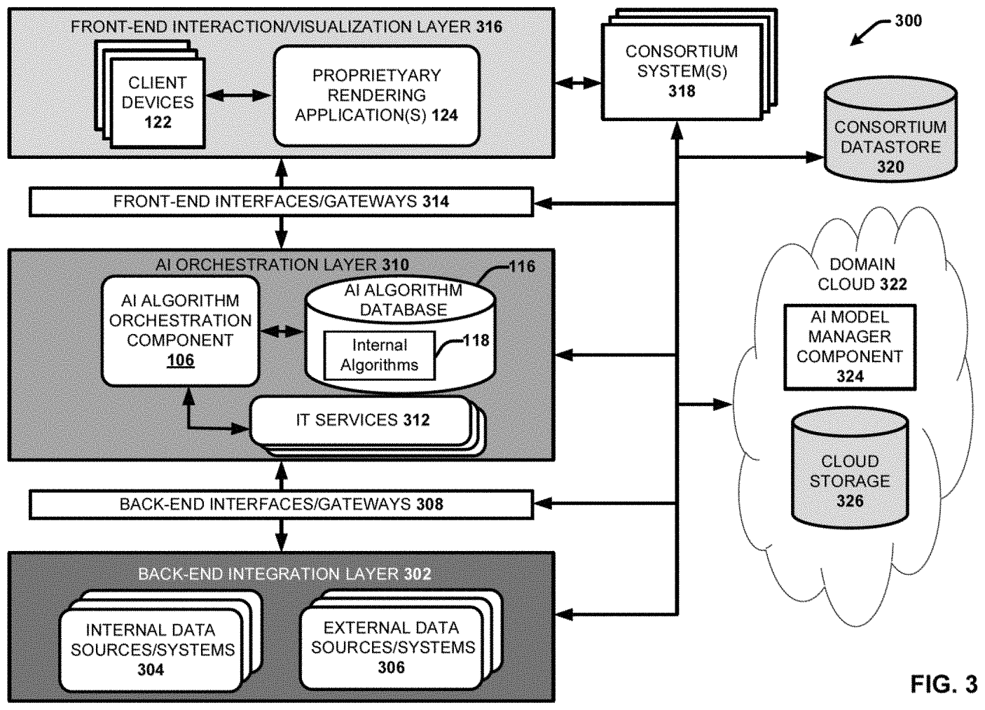

FIG. 3 illustrates a block diagram of an example, non-limiting system 300 that facilitates integrating AI informatics into systems of various domains using a distributed deployment architecture, in accordance with one or more embodiments of the disclosed subject matter. In this regard, system 300 provides an example an example system architecture via which system 100 and/or system 200 can be implemented. Repetitive description of like elements employed in respective embodiments is omitted for sake of brevity.

With reference to FIGS. 1-3, in various embodiments, the distributed deployment architecture of system 300 can include three interconnected layers including a back-end integration layer 302, an AI orchestration layer 310, and a front-end interaction/visualization layer 316. The back-end integration layer 102 can be coupled to the AI orchestration layer 110 via one or more back-end interfaces/gateways 308, and the front-end interaction/visualization layer 316 can be coupled to the AI orchestration layer via one or more front-end interfaces/gateways 314.

The back-end integration layer 302 can comprise various disparate data sources and systems that provide data and/or services associated with the industry/domain in which system 100 is applied to facilitate securely deploying AI models and distributing inferences generated therefrom. For example, in implementations in which system 300 is applied to healthcare, the back-end integration layer 302 can comprise various disparate healthcare data sources and healthcare systems that provide healthcare data and/or services associated with generating or consuming the healthcare data. The back-end integration layer 302 can comprise one or more internal data sources/systems 304, as well as one or more external data sources/systems 306 that provide the input data (e.g., input data 102) for processing by the one or more internal algorithms 118.

For example, as applied to healthcare, the one or more internal data sources/systems 304, as well as one or more external data sources/systems 306 that provide the appropriate input data for processing by one or more clinical inferencing models, including medical image inferencing models. In this regard, the one or more internal and/or external data sources/systems can include medical imaging systems configured to provide medical image data, patient records systems configured to provide patient health information, medical reporting systems configured to provide patient pathology reports, and the like. The back-end integration layer 302 can also comprise healthcare reporting/monitoring systems that provide real-time (or substantially real-time) contextual input information associated with patients, clinicians, and medical supplies/instruments at one or more healthcare facility (e.g., physiological information, location information, workflow information, etc.) that can be used as input when the healthcare models are run in association with performance of actual clinical and/or operational workflows in real-time to enhance performance of these workflows with AI oversight. Various embodiments, the data elements and input data included in the back-end integration layer 302 are searchable and can be accessed by multiple systems (investigators) and customers in association with instructions and/or request to apply one or more of the internal algorithms 118 thereto.

In the embodiment shown, internal data sources/systems 304 are distinguished from external data sources/systems 306 to indicate that the internal data sources/systems 304 can comprise one or more proprietary data sources/systems associated with the enterprise or entity that owns and/or controls the internal algorithms 118 provided in the AI algorithms database 116 as wells as the proprietary rendering application(s) 124. The external data sources/systems can include any input data source associated with a third-party entity that does not own, manage or control the internal algorithms 118 provided in the AI algorithms database 116 and/or the proprietary rendering application(s) 124 In this regard, the back-end integration layer 302 can amass various healthcare data sources and systems provided by different enterprises/entities at various locations around the world (and/or in the cloud) to enable access to all types of data that can be used as input to the internal algorithms 118.

The AI orchestration layer 310 can provide various processing functions associated with securely provisioning the internal algorithms 118 to consuming applications and systems in various domains/industries. In this regard, the AI orchestration layer 310 can further facilitate integrating and applying the one or more internal AI models into actual industry operational workflows in a manner that protects the integrity of the AI models (e.g., prevents piracy of the models). In the embodiment shown, the AI orchestration layer 310 can include the AI algorithm orchestration component 106, the AI algorithm database 116 and one or more other IT services 312.

As described above with reference to system 100, the AI algorithm database 116 can provide one or more internal algorithms (e.g., AI models) configured to provide AI informatics associated with various domains/industries. For example, as applied to healthcare, the AI algorithm database 116 can provide one or more healthcare AI models configured to provide AI informatics associated with various healthcare domains. In this regard, the models included in the AI algorithm database 116 can be configured to process various types of healthcare input data provided by the back-end integration layer 302 to generate various clinical and/or operational inferences, determinations, evaluations, recommendations, etc., based on the input data. For example, the models included in the AI algorithm database 116 can include one or more diagnostic models configured to generate diagnostic information based on medical image and signal data, patient physiological information (e.g., including signs/symptoms), patient medical history/record information, patient laboratory information/test results, patient omics data, and the like.

In another example, the models included AI algorithm database 116 can include medical ordering models configured to determine information regarding how to treat a patient based on a diagnosis of the patient, a current state of the patient, medical history of the patient, signs and symptoms, demographic information, insurance information and the like. For instance, the ordering models can generate information regarding recommended medical tests/studies to be performed, procedures to be performed, medications to administer, physical activities to perform (or not perform) rehabilitation/therapy activities to perform, dietary restrictions, etc. In another example, the AI algorithm database 116 can include one or more healthcare AI models configured to generate information regarding recommended courses of care for specific patients and specific clinical and/or operational contexts that facilitate optimal clinical and/or financial outcomes. The AI models can also include models configured to identify clinical and operational variances in workflows relative to standard operating procedures (SOPs), clinical complications, responses to remediate clinical complications, and the like. In another example, the AI models can include models configured to provide recommendations regarding assigning clinicians to specific patients (e.g., based on learned performance and competency levels of the clinician, the condition of the patient, preferences of the clinician preferences of the patient, etc.). In another example, the AI models can include one or more models configured to determine recommendations regarding how to prioritize placement of patients to a limited number of beds in a medical facilitate based on patient needs, a state of the healthcare facility, predicted occupancy levels and the like.

In this regard, the types of outputs generated by the AI models included in the AI algorithm database 116, the number of models and the diversity of the models can vary. It should be appreciated that a wide array of sophisticated healthcare AI informatics and associated models are possible given the granularity, diversity and massive amount of healthcare data that can be provided by the back-end integration layer 302, as well as the distributed deployment functionality afforded by system 300 as discussed herein. In this regard, the various example AI models discussed above merely provide a small taste of the possible applications of AI informatics in healthcare that can be enabled by the distributed deployment architecture of system 300. The distributed deployment architecture of system 300 is also designed such that existing models included in the AI algorithm database 116 can be regularly updated and refined, and new models can be added to the AI algorithm database 116 over time as new input data is received, new technology arises, and the like.

The AI orchestration layer 310 can be coupled to the back-end integration layer 302 via one or more back-end interfaces/gateways 308. The one or more back-end interfaces/gateways 308 can provide flexible interfaces allowing for the AI model input data (e.g., input data 102) to be extracted and/or otherwise received from its network source location by the AI algorithm orchestration component 106. For example, in one or more embodiments, the back-end interfaces/gateways 308 can provide domain and site-specific interfaces to back-end enterprise systems and sources (e.g., as an adapter/abstraction layer). In this regard, the AI orchestration layer 310 can enable access to AI model input data from curated data repositories provided by the various disparate data sources of the back-end integration layer 302.

In various embodiments, the AI algorithm orchestration component 106 can provide the analytical tools for securely applying the internal algorithms 118 to facilitate enhancing clinical and operational workflows of various healthcare systems. In this regard, the AI algorithm orchestration component 106 can provide interoperability to assert the healthcare AI models included in the AI algorithm database 116 as part of clinical routine. For example, in one or more embodiments, the AI algorithm orchestration component 106 can interface with one or more proprietary rendering applications 124 employed by a healthcare organization to facilitate clinical and/or operational workflows of the healthcare organization. For instance, as discussed above with reference to system 100 and system 200, the proprietary rendering applications 124 can a a medical imaging visualization/interaction application configured to provide functions associated with viewing and evaluating medical images by physicians (or patients, or other appropriate entities). According to this example, the AI algorithm orchestration component 106 can facilitate identifying and calling one or more applicable AI models from the AI algorithm database 116 in association with usage of the imaging application by a clinician and applying the one or more AI models to generate to the designated input data (which may be provided by the client device 122 and/or the back-end integration layer 302). The AI algorithm orchestration component 106 can further facilitate providing the encrypted/encoded AI model results to the clinician physician via the proprietary rendering application 124 during the clinical workflow.

The AI orchestration layer 310 can also include one or more additional IT services 318 that can provide various services associated with integrating one or more features and functionalities of the AI orchestration layer 110 with other systems, devices (e.g., the client devices 122) and/or applications associated with the front-end interaction/visualization layer 316 and the back-end integration layer 302. For example, the one or more additional IT services 312 can facilitate defining and applying internal algorithm usage agreements between the internal algorithm provider and consuming entities/applications. For example, the internal algorithm usage agreements can specify the input data to be processed by the one or more internal algorithms 118, the source of the input data, the frequency/time with which to process the input data, the specific algorithms to apply, the specific client devices 122 and/or proprietary rendering application 124 (or another destination) to provide the encrypted model output data, and so on. The one or more additional IT services 312 can also facilitate querying the models included in the AI algorithm database 116, as well as data elements and input data included in the back-end integration layer 302 by multiple systems (investigators) in association with automatically extracting the appropriate input data for processing by the internal algorithms 118. The AI orchestration layer 310 can thus maximize reuse and sharing of the internal algorithms in a secure manner that scales across different enterprises, enterprise departments and regions.

The front-end visualization layer 316 can facilitate user interaction in association with consuming data and services provided by the back-end integration layer 302 and/or the AI orchestration layer 310. In the embodiment shown, the front-end visualization layer 122 can include the one or more client devices 122 and the one or more proprietary rendering applications 124. The client devices 124 can include various types of devices associated with users of system 300 via which the users can consume data and services provided by system 300 in association with usage of the one or more proprietary rendering applications 124 including the interoperability to consume the decrypted output data provided by the AI orchestration layer 310 and prevent or minimize its exportation to unauthorized systems/device. For example, some suitable client devices 123 can include but are not limited to a desktop computer, a laptop computer, a television, an Internet enabled television, a mobile phone, a smartphone, a tablet personal computer (PC), or a personal digital assistant (PDA), a heads-up display (HUD), an augmented reality (AR) device, a virtual reality (VR) device, a wearable device, an implanted medical device (IMD), a medical instrument or device capable of receiving and applying computer readable instructions (e.g., an imaging device, robotics instrument, etc.), and the like. In the various embodiments, one or more of the client devices 122 can be configured to provide for rending the decrypted output data 140 to a user of the client device using as suitable output component, such as a display, a speaker, a sensory or haptic feedback device, and the like.

The term user or end-user as used herein can refer to any entity that uses data and/or services provided by system 300. In this regard, a user or end-user can include human users as well as machines, devices, and systems. For example, in some contexts, an end-user can include a person that uses the proprietary rendering application 124 to send receive and consume (e.g., view, interact with, etc.), decrypted AI model output data generated by the internal algorithms 118. In other contexts, the end-user can include another application or system that uses the decrypted AI model output data for other post-processing tasks.

The manner of deployment of the proprietary rendering applications 124 can vary. For example, in some embodiments the proprietary rendering applications 124 can comprise one or more web-applications. For example, in some implementations, the web-applications can include angular-based applications. In other implementations, the web-applications can be non-angular web applications. As a web-application, the AI the proprietary rendering applications 124 can comprise their own cod-based binary/object code. In other embodiments, the proprietary rendering applications 124 can comprise one or more cloud-applications, one or more thin client applications, one or more thick client applications, one or more native client applications, one or more hybrid client applications, or the like. In this regard, although the proprietary rendering applications 124 shown as being part of the front-end interaction/visualization layer 316, in other embodiments, the proprietary rendering applications 124 can be located in the AI orchestration layer 310, the back-end integration layer 302 and/or the in the domain cloud 322.

In this regard, in some embodiments, in addition to the three primary layers (e.g., the back-end integration layer 302, the AI orchestration layer 310, and the front-end interaction/visualization layer 316), system 300 can also include one or more consortium systems 318, a consortium data store 320 and a domain cloud 322. These additional components of system 300 can be communicatively coupled to one another and the respectively layers to facilitate sharing of information and services between the respective components of system 300.

The consortium systems 318 can include partner applications that can be provided with the healthcare AI models included in the AI algorithm database 116 in accordance with the techniques described herein. The consortium systems 318 can also provide additional data sets for model training and development. The consortium datastore 320 can compile information and data provided by the consortium systems 318 as well as the information developed by the AI orchestration layer for efficient and seamless access and utilization.

The domain cloud 322 can provide anther means for combining relevant domain data and services from multiple sources into a network accessible cloud-based environment. For example, in embodiments in which system 300 is employed to facilitate integrating AI informatics into various healthcare systems, the domain cloud 332 can correspond to a health cloud. For example, the domain cloud 322 can include cloud storage 326 that combines electronic medical records, medical device data, even wearables, into a single location. In some embodiments, the input data 102 and the encrypted output data 120 can also be stored in the cloud storage 326. The domain cloud 132 can also include an AI model manager component 324 that can facilitate identifying, accessing and employing appropriate AI healthcare models included in the AI algorithm database 116 by the consortium systems 318.

The deployment architecture 300 can vary. However, it should be appreciated that although not shown, the various systems, services, devices, applications, components and the like, of system 300, can be coupled to at least one memory that stores the computer executable components or the front-end interaction/visualization layer 316, the AI orchestration layer 310, and the back-end integration layer 302. Further the various systems, services, devices, applications, components and the like, of system 300 can be communicatively coupled to at least one processor that executes the computer executable components. In various embodiments, one or more of the various systems, services, devices, applications, components and the like, of system 300 can be deployed in a cloud architecture, a virtualized enterprise architecture, or an enterprise architecture wherein one the front-end components and the back-end components are distributed in a client/server relationship. With these embodiments, the features and functionalities of one or more of the various systems, services, devices, applications, components and the like, of system 300 can be deployed a web-application, a cloud-application, a thin client application, a thick client application, a native client application, a hybrid client application, or the like, wherein one or more of the front-end components are provided at a client device 122 (e.g., a mobile device, a laptop computer, a desktop computer, etc.) and one or more of the back-end components are provided in the cloud, on a virtualized server, a virtualized data store, a remote server, a remote data store, a local data center, etc., and accessed via a network (e.g., the Internet). Various example deployment architectures for system 100 are described infra with reference to FIGS. 7-12.

While one or more systems, services, devices, applications, components and the like, of system 300 are illustrated as separate components, it is noted that the various components can be comprised of one or more other components. Further, it is noted that the embodiments can comprise additional components not shown for sake of brevity. Additionally, various aspects described herein may be performed by one device or two or more devices in communication with each other. For example, one or more systems, services, devices, applications, components and the like, of system 300 can be located on separate (real or virtual) machines and accessed via a network (e.g., the Internet).

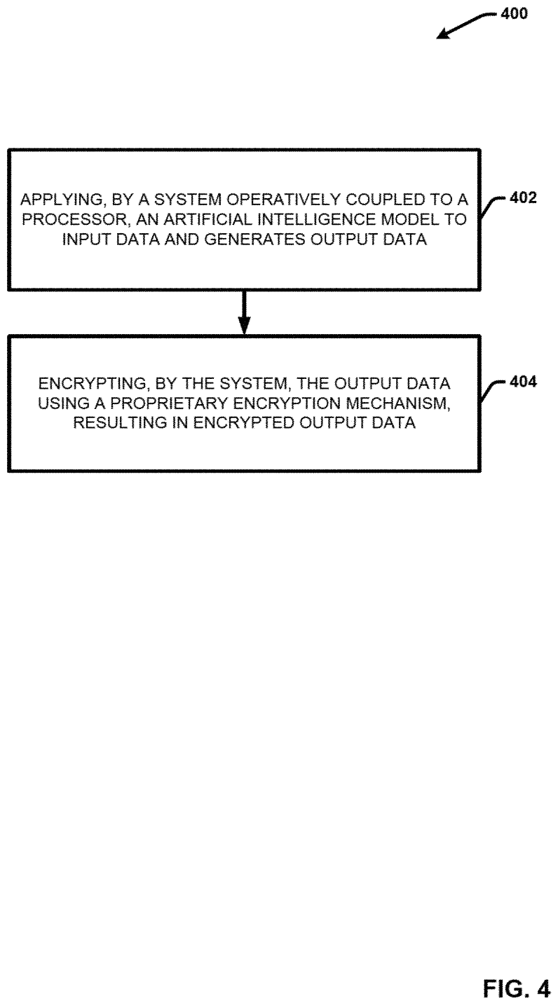

FIG. 4 illustrates a block diagram of an example, non-limiting computer implemented method 400 that facilitates securely deploying AI models and distributing inferences generated therefrom, in accordance with one or more embodiments of the disclosed subject matter. Repetitive description of like elements employed in respective embodiments is omitted for sake of brevity.

In accordance with method 400, at 402 a system operatively coupled to a processor (e.g., system 100, system 200, system 300 and the like), can apply an AI model to input data (e.g., input data 102) and generate output data (e.g., output data 111). At 404, the system can encrypt the output data using a proprietary encryption mechanism (e.g., via the encryption component 114), resulting in encrypted output data (e.g., encrypted output data 120).

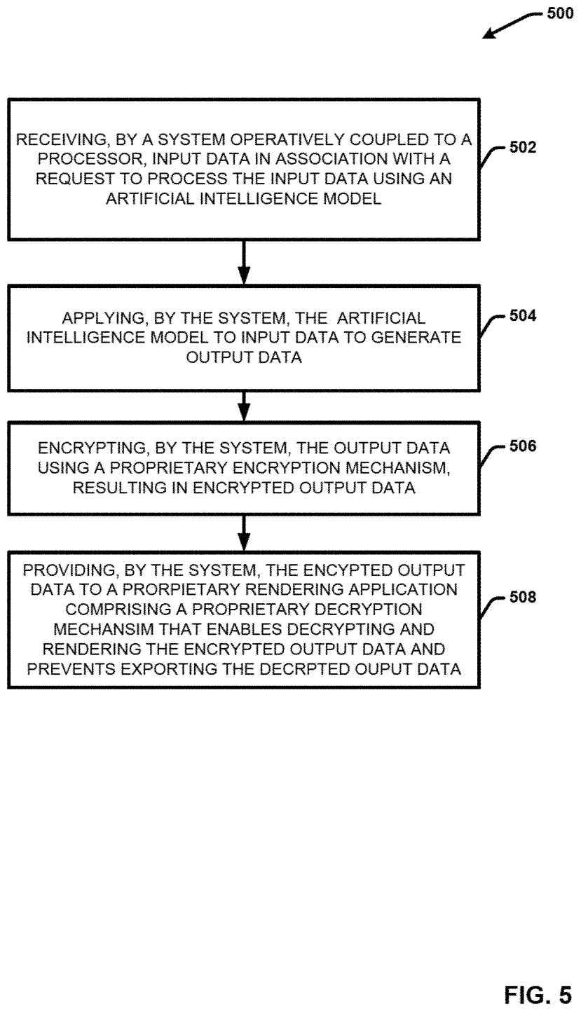

FIG. 5 illustrates a block diagram of another example, non-limiting computer implemented method 500 that facilitates securely deploying AI models and distributing inferences generated therefrom, in accordance with one or more embodiments of the disclosed subject matter. Repetitive description of like elements employed in respective embodiments is omitted for sake of brevity.

In accordance with method 500, at 502 a system operatively coupled to a processor (e.g., system 100, system 200, system 300 and the like) can receive input data (e.g., input data 102 received via reception component 108) in association with a request to process the input data using an AI model. For example, with reference to FIGS. 1 and 3, in some implementations the request can be generated by the request component 126 using the proprietary rendering application 124. In other embodiments, the request can be provided by another device or system (e.g., one or more interna data sources systems, one or more external data sources/systems, and/or one or more consortium systems). In some embodiments, the request can include the input data 102. In other embodiments, the request can identify or indicate the input data 102 and include information that enables the AI algorithm orchestration component 106 to identify and extract the input data 102 (e.g., from the back-end integration layer 302, the consortium datastore 320 and/or the cloud storage 326). The request can further identify or indicate the specific AI model to apply to the input data. Additionally, or alternatively, the AI algorithm orchestration component 106 and/or the AI model manager 324 can select the appropriate AI models to apply to the input data based on the input data, the models available and/or one or more internal algorithm usage agreements defined between the requesting entity and the internal algorithm provider.

At 504, the system can apply the AI model to input data (e.g., input data 102) and generate output data (e.g., output data 111). At 506, the system can encrypt the output data using a proprietary encryption mechanism (e.g., via the encryption component 114), resulting in encrypted output data (e.g., encrypted output data 120). At 508, the system can further provide the encrypted output data to a proprietary rendering application (e.g., proprietary rendering application 124) comprising a proprietary decryption mechanism that enables decrypting and rendering the encrypted output data and prevents exporting the decrypted output data.

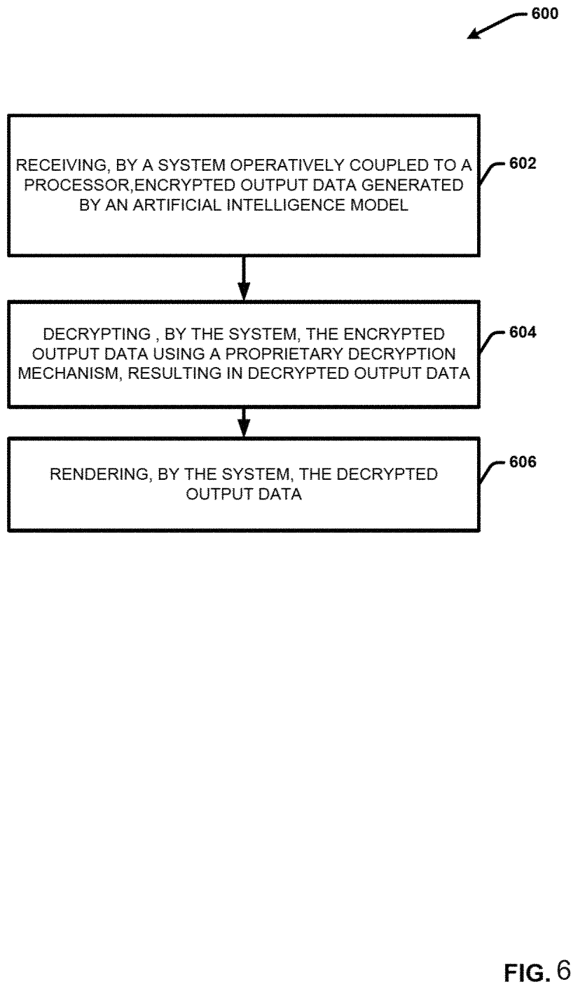

FIG. 6 illustrates a block diagram of another example, non-limiting computer implemented method 600 that facilitates securely deploying AI models and distributing inferences generated therefrom, in accordance with one or more embodiments of the disclosed subject matter. Repetitive description of like elements employed in respective embodiments is omitted for sake of brevity.

In accordance with method 600, at 602 a system operatively coupled to a processor (e.g., system 100, system 200, system 300 and the like) can receive (e.g., via client device 122 and/or a proprietary rendering application 124) encrypted output (e.g., encrypted output data 120) data generated by an AI model (e.g., one or more of the internal algorithms 118). At 604, the system can decrypt (e.g., via the decryption component 134) the encrypted output data using a proprietary decryption mechanism, resulting in decrypted output data (e.g., decrypted output data 140). At 604, the system can render the decrypted output data (e.g., via rendering component 130 and/or output device 138).

One or more embodiments can be a system, a method, and/or a computer program product at any possible technical detail level of integration. The computer program product can include a computer readable storage medium (or media) having computer readable program instructions thereon for causing a processor to carry out one or more aspects of the present embodiments.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the entity's computer, partly on the entity's computer, as a stand-alone software package, partly on the entity's computer and partly on a remote computer or entirely on the remote computer or server. In the latter scenario, the remote computer can be connected to the entity's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It can be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession may, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In connection with FIG. 7, the systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which can be explicitly illustrated herein.

With reference to FIG. 7, an example environment 700 for implementing various aspects of the claimed subject matter includes a computer 702. The computer 702 includes a processing unit 704, a system memory 706, a codec 735, and a system bus 708. The system bus 708 couples system components including, but not limited to, the system memory 706 to the processing unit 704. The processing unit 704 can be any of various available processors. Dual microprocessors and other multiprocessor architectures also can be employed as the processing unit 704.

The system bus 708 can be any of several types of bus structure(s) including the memory bus or memory controller, a peripheral bus or external bus, or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1374), and Small Computer Systems Interface (SCSI).

The system memory 706 includes volatile memory 710 and non-volatile memory 712, which can employ one or more of the disclosed memory architectures, in various embodiments. The basic input/output system (BIOS), containing the basic routines to transfer information between elements within the computer 702, such as during start-up, is stored in non-volatile memory 712. In addition, according to present innovations, codec 735 can include at least one of an encoder or decoder, wherein the at least one of an encoder or decoder can consist of hardware, software, or a combination of hardware and software. Although, codec 735 is depicted as a separate component, codec 735 can be contained within non-volatile memory 712. By way of illustration, and not limitation, non-volatile memory 712 can include read only memory (ROM), programmable ROM (PROM), electrically programmable ROM (EPROM), electrically erasable programmable ROM (EEPROM), Flash memory, 3D Flash memory, or resistive memory such as resistive random access memory (RRAM). Non-volatile memory 712 can employ one or more of the disclosed memory devices, in at least some embodiments. Moreover, non-volatile memory 712 can be computer memory (e.g., physically integrated with computer 702 or a mainboard thereof), or removable memory. Examples of suitable removable memory with which disclosed embodiments can be implemented can include a secure digital (SD) card, a compact Flash (CF) card, a universal serial bus (USB) memory stick, or the like. Volatile memory 710 includes random access memory (RAM), which acts as external cache memory, and can also employ one or more disclosed memory devices in various embodiments. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM) and so forth.

Computer 702 can also include removable/non-removable, volatile/non-volatile computer storage medium. FIG. 7 illustrates, for example, disk storage 714. Disk storage 714 includes, but is not limited to, devices like a magnetic disk drive, solid state disk (SSD), flash memory card, or memory stick. In addition, disk storage 714 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 714 to the system bus 708, a removable or non-removable interface is typically used, such as interface 716. It is appreciated that disk storage 714 can store information related to an entity. Such information might be stored at or provided to a server or to an application running on an entity device. In one embodiment, the entity can be notified (e.g., by way of output device(s) 736) of the types of information that are stored to disk storage 714 or transmitted to the server or application. The entity can be provided the opportunity to opt-in or opt-out of having such information collected or shared with the server or application (e.g., by way of input from input device(s) 728).

It is to be appreciated that FIG. 7 describes software that acts as an intermediary between entities and the basic computer resources described in the suitable operating environment 700. Such software includes an operating system 718. Operating system 718, which can be stored on disk storage 714, acts to control and allocate resources of the computer system 702. Applications 720 take advantage of the management of resources by operating system 718 through program modules 724, and program data 726, such as the boot/shutdown transaction table and the like, stored either in system memory 706 or on disk storage 714. It is to be appreciated that the claimed subject matter can be implemented with various operating systems or combinations of operating systems.

An entity enters commands or information into the computer 702 through input device(s) 728. Input devices 728 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 704 through the system bus 708 via interface port(s) 730. Interface port(s) 730 include, for example, a serial port, a parallel port, a game port, and a universal serial bus (USB). Output device(s) 736 use some of the same type of ports as input device(s) 728. Thus, for example, a USB port can be used to provide input to computer 702 and to output information from computer 702 to an output device 736. Output adapter 734 is provided to illustrate that there are some output devices 736 like monitors, speakers, and printers, among other output devices 736, which require special adapters. The output adapters 734 include, by way of illustration and not limitation, video and sound cards that provide a means of connection between the output device 736 and the system bus 708. It should be noted that other devices or systems of devices provide both input and output capabilities such as remote computer(s) 738.

Computer 702 can operate in a networked environment using logical connections to one or more remote computers, such as remote computer(s) 738. The remote computer(s) 738 can be a personal computer, a server, a router, a network PC, a workstation, a microprocessor based appliance, a peer device, a smart phone, a tablet, or other network node, and typically includes many of the elements described relative to computer 702. For purposes of brevity, only a memory storage device 740 is illustrated with remote computer(s) 738. Remote computer(s) 738 is logically connected to computer 702 through a network interface 742 and then connected via communication connection(s) 744. Network interface 742 encompasses wire or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 744 refers to the hardware/software employed to connect the network interface 742 to the bus 708. While communication connection 744 is shown for illustrative clarity inside computer 702, it can also be external to computer 702. The hardware/software necessary for connection to the network interface 742 includes, for exemplary purposes only, internal and external technologies such as, modems including regular telephone grade modems, cable modems and DSL modems, ISDN adapters, and wired and wireless Ethernet cards, hubs, and routers.

The illustrated aspects of the disclosure may also be practiced in distributed computing environments where certain tasks are performed by remote processing devices that are linked through a communications network. In a distributed computing environment, program modules can be located in both local and remote memory storage devices.

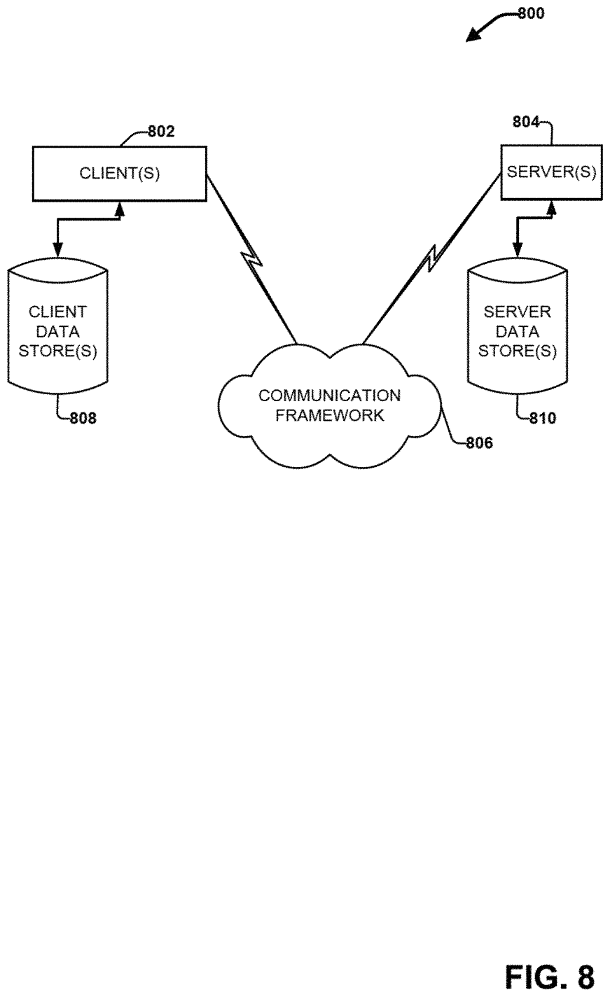

Referring to FIG. 8, there is illustrated a schematic block diagram of a computing environment 800 in accordance with this disclosure in which the subject systems (e.g., systems 100, 200, 300 and the like), methods and computer readable media can be deployed. The computing environment 800 includes one or more client(s) 802 (e.g., laptops, smart phones, PDAs, media players, computers, portable electronic devices, tablets, and the like). The client(s) 802 can be hardware and/or software (e.g., threads, processes, computing devices). The computing environment 800 also includes one or more server(s) 804. The server(s) 804 can also be hardware or hardware in combination with software (e.g., threads, processes, computing devices). The servers 804 can house threads to perform transformations by employing aspects of this disclosure, for example. In various embodiments, one or more components, devices, systems, or subsystems of system 100, 200 and 300, can be deployed as hardware and/or software at a client 802 and/or as hardware and/or software deployed at a server 804. One possible communication between a client 802 and a server 804 can be in the form of a data packet transmitted between two or more computer processes wherein the data packet may include healthcare related data, training data, AI models, input data for the AI models, encrypted output data generated by the AI models, and the like. The data packet can include a metadata, e.g., associated contextual information, for example. The computing environment 800 includes a communication framework 806 (e.g., a global communication network such as the Internet, or mobile network(s)) that can be employed to facilitate communications between the client(s) 802 and the server(s) 804.

Communications can be facilitated via a wired (including optical fiber) and/or wireless technology. The client(s) 802 include or are operatively connected to one or more client data store(s) 808 that can be employed to store information local to the client(s) 802 (e.g., associated contextual information). Similarly, the server(s) 804 are operatively include or are operatively connected to one or more server data store(s) 810 that can be employed to store information local to the servers 804 (e.g., native medical image data 132, indexed medical image data information 134, and the like).

In one embodiment, a client 802 can transfer an encoded file, in accordance with the disclosed subject matter, to server 804. Server 804 can store the file, decode the file, or transmit the file to another client 802. It is to be appreciated, that a client 802 can also transfer uncompressed file to a server 804 can compress the file in accordance with the disclosed subject matter. Likewise, server 804 can encode video information and transmit the information via communication framework 806 to one or more clients 802.

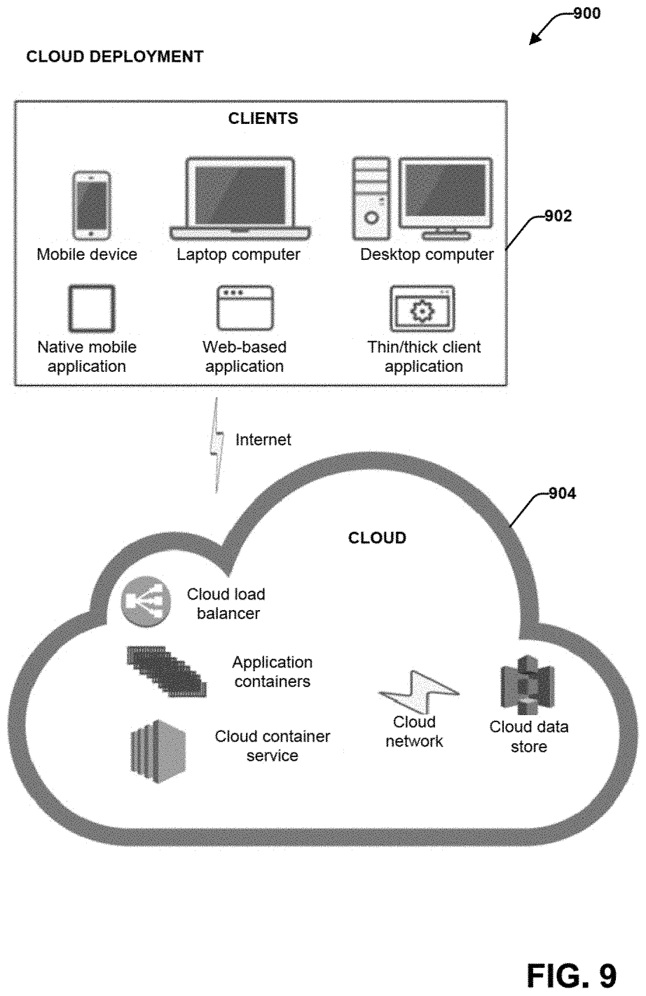

FIG. 9 illustrates a schematic block diagram of another example computing environment 900 in accordance with this disclosure in which the subject systems (e.g., systems 100, 200, 300 and the like), methods and computer readable media can be deployed. The computing environment 900 includes a cloud deployment architecture consisting of one or more clients 902 that can be communicatively coupled to a system cloud 904 via a network (e.g., the Internet). The system cloud 904 can include a cloud load balances, one or more application container, one or more cloud service containers, a cloud data store, and a cloud network that communicatively couples the one or more cloud components to the cloud data store. In accordance with the cloud deployment architecture, the clients 902 can include one or more clients devices (e.g., a mobile device, a laptop computer, a desktop computer, etc.) which can include or employ a suitable application (e.g., a native mobile application, a web-based application, a thin/thick client application, etc.) to access and employ one or more features and functionalities of the subject systems (e.g., system 100, 200, 300 and the like) deployed in the system cloud 904. In various implementations, the one or more components of systems 100, 200 and/or 300 can be distributed between the clients 902 and the system cloud 904.

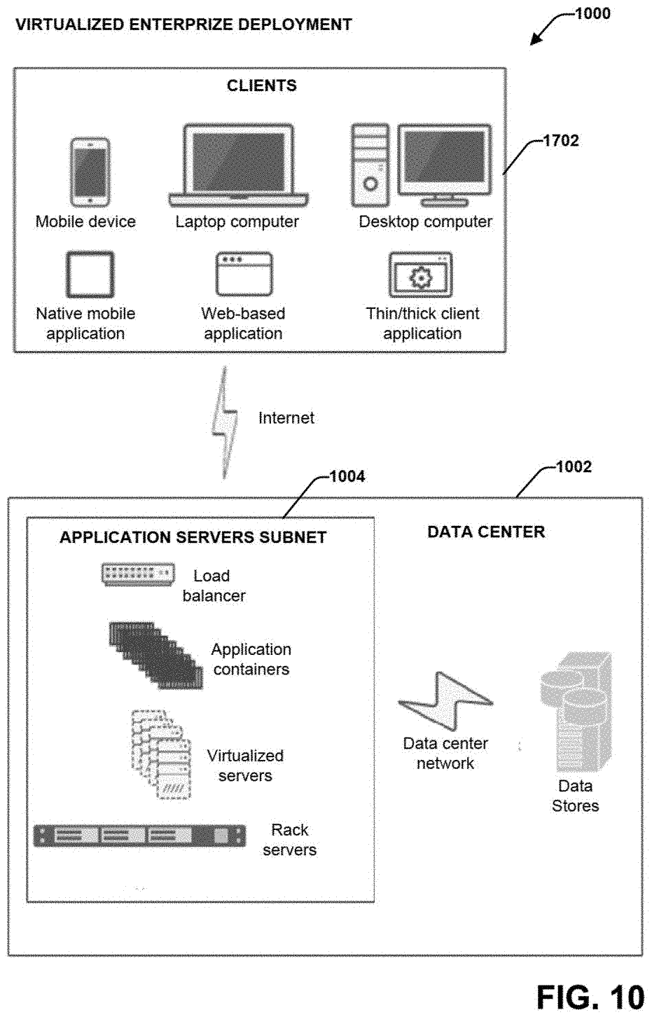

FIG. 10 illustrates a schematic block diagram of another example computing environment 1000 in accordance with this disclosure in which the subject systems (e.g., systems 100, 200, 300 and the like), methods and computer readable media can be deployed. The computing environment 1000 includes a virtualized enterprise deployment consisting of one or more clients 902 that can be communicatively coupled to a remote data center 1002 via a network (e.g., the Internet). The remote data center 1002 can include an application servers subnet 1004 which can provide a load balancer, one or more application containers, one or more virtualized servers and one or more rack servers. The data center 1002 can also include one or more data stores that can be communicatively coupled to the application servers subnet 1004 via a data center network. In accordance with the virtualized enterprise deployment, the clients 902 can include one or more clients devices (e.g., a mobile device, a laptop computer, a desktop computer, etc.) which can include or employ a suitable application (e.g., a native mobile application, a web-based application, a thin/thick client application, etc.) to access and employ one or more features and functionalities of the subject distributed AI model deployment systems (e.g., system 100, 200, 300 and the like) deployed in the data center 1002 and application servers subnet 1004. In various implementations, the one or more components of systems 100, 200 and/or 300 can be distributed between the clients 902 and the application servers subnet 1004 and the data center 1002.

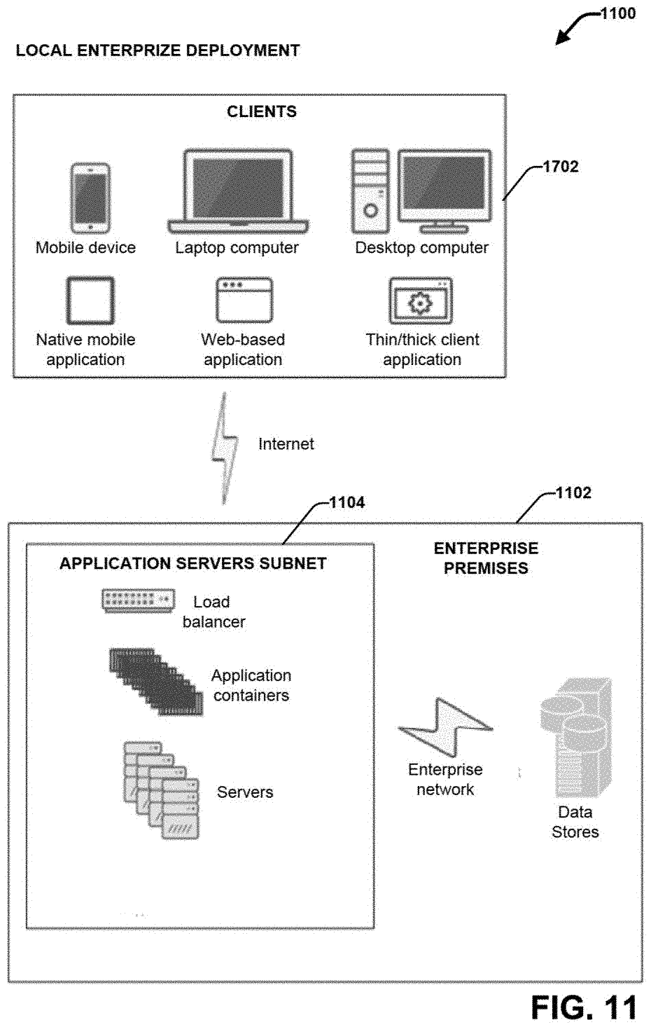

FIG. 11 illustrates a schematic block diagram of another example computing environment 1100 in accordance with this disclosure in which the subject systems (e.g., systems 100, 200, 300 and the like), methods and computer readable media can be deployed. The computing environment 1100 includes a local enterprise deployment consisting of one or more clients 902 that can be communicatively coupled to an application servers subnet 1104 via a network (e.g., the Internet). In accordance with this embodiment, the application servers subnet 1104 can be provided at the enterprise premises 1102 (e.g., as opposed to a remote data center 1002). The application servers subnet 1104 can include a load balancer, one or more application containers and one or more servers. The application servers subnet 1104 can be communicatively coupled to one or more data stores provided at the enterprise premises 1102 via an enterprise network. Similar to the cloud and virtualized enterprise deployments, the clients 902 can include one or more clients devices (e.g., a mobile device, a laptop computer, a desktop computer, etc.) which can include or employ a suitable application (e.g., a native mobile application, a web-based application, a thin/thick client application, etc.) to access and employ one or more features and functionalities of the subject distributed AI model deployment systems (e.g., system 100, 200, 300 and the like) deployed at the enterprise premises 1102 and the application servers subnet 1104. In various implementations, the one or more components of systems 100, 200 and/or 300 can be distributed between the clients 902 and the application servers subnet 1104 and the enterprise premises 1102.

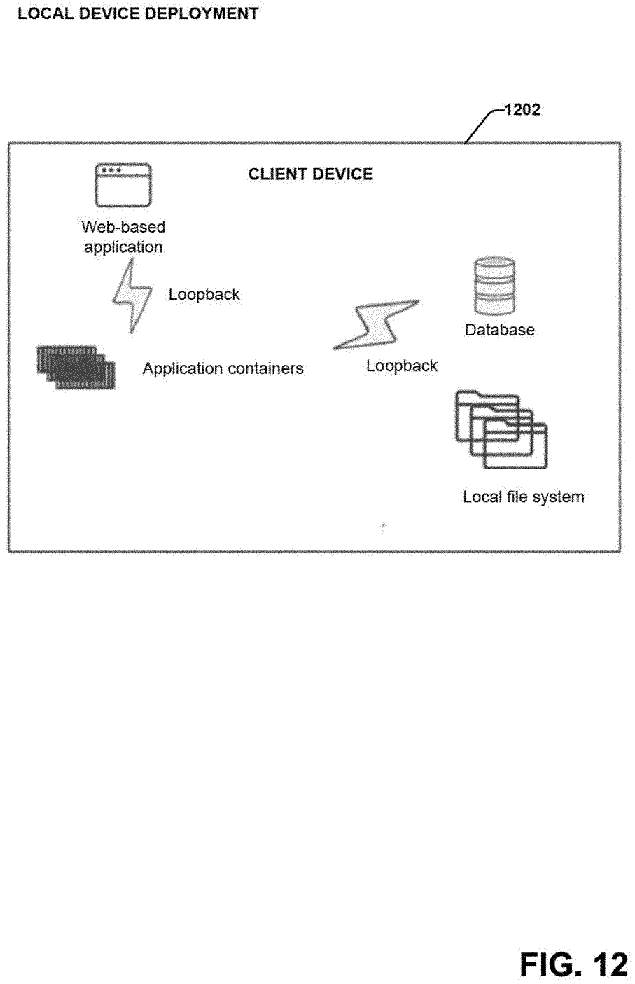

FIG. 12 illustrates a schematic block diagram of another example computing environment 1200 in accordance with this disclosure in which the subject systems (e.g., systems 100, 200, 300 and the like), methods and computer readable media can be deployed. The computing environment 1200 includes a local device deployment in which all of the components of system 100, 200 and/or 300 are provided at a single client device 1202. With this implementation, the client device 1202 can include a web-based application which can be communicatively coupled via a loopback to one or more application containers. The one or more application containers can be communicatively coupled via a loopback to one or more databases and/or one or more local file systems.

While the subject matter has been described above in the general context of computer-executable instructions of a computer program product that runs on a computer and/or computers, those skilled in the art will recognize that this disclosure also can or can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments where tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “subsystem” “platform,” “layer,” “gateway,” “interface,” “service,” “application,” “device,” and the like, can refer to and/or can include one or more computer-related entities or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration and are intended to be non-limiting. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of entity equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The descriptions of the various embodiments have been presented for purposes of illustration, but are not intended to be exhaustive or limited to the embodiments disclosed. Many modifications and variations can be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
a memory that stores computer executable components; and
a processor that executes the computer executable components stored in the memory, wherein the computer executable components comprise:
an algorithm execution component that applies an artificial intelligence model to input data and generates output data; and
an encryption component that encrypts the output data using a proprietary encryption mechanism, resulting in encrypted output data.

2. The system of claim 1, wherein the proprietary encryption mechanism prevents usage and rendering of the encrypted output data without decryption of the encrypted output data using a proprietary decryption mechanism.

3. The system of claim 2, wherein the computer executable components further comprise:
a decryption component that decrypts the encrypted output data using the proprietary decryption mechanism to obtain the output data; and
a rendering component that renders the output data.

4. The system of claim 3, wherein the decryption component and the rendering component are included in a proprietary software application, and wherein the proprietary software application prevents exporting of the output data.

5. The system of claim 4, wherein the computer executable components further comprise:
an orchestration component that provides the encrypted output data to the proprietary software application.

6. The system of claim 5, wherein the orchestration component receives the input data from an external system in association with a request to process the input data via the artificial intelligence model.

7. The system of claim 4, wherein the proprietary software application comprises a web-application accessible to external systems via one or more wireless communication networks.

8. The system of claim 4, wherein the proprietary software application comprises a medical image viewer application.

9. The system of claim 8, wherein the input data comprises medical image data and wherein the output data comprises modified medical image data.

10. The system of claim 9, wherein the proprietary encryption mechanism comprises embedding one or more proprietary tags within the output data, the one or more private tags formatted according to the digital imaging and communications in medicine (DICOM) format.

11. The system of claim 1, wherein the artificial intelligence model comprises a clinical inferencing model.

12. A method, comprising:
applying, by a system operatively coupled to a processor, an artificial intelligence model to input data and generates output data; and
encrypting, by the system, the output data using a proprietary encryption mechanism, resulting in encrypted output data.

13. The method of claim 12, wherein the proprietary encryption mechanism prevents usage and rendering of the encrypted output data without decryption of the encrypted output data using a proprietary decryption mechanism.

14. The method of claim 13, further comprising:
decrypting, by the system, the encrypted output data using the proprietary decryption mechanism to obtain the output data; and
rendering, by the system, the output data.

15. The method of claim 13, further comprising:
providing, by the system, the encrypted output data to a proprietary software application comprising the proprietary encryption mechanism, wherein the proprietary software application prevents exporting of the output data.

16. The method of claim 13, wherein the proprietary software application comprises a web-application accessible to external systems via one or more wireless communication networks.

17. The method of claim 12, further comprising:
receiving, by the system, the input data from an external system in association with a request to process the input data via the artificial intelligence model, wherein the applying is responsive to the receiving.

18. A non-transitory machine-readable storage medium, comprising executable instructions that, when executed by a processor, facilitate performance of operations, comprising:
receiving encrypted output data generated by an artificial intelligence model;
decrypting the encrypted output data using a proprietary decryption mechanism, resulting in decrypted output data; and
rendering the decrypted output data.

19. The non-transitory machine-readable storage medium of claim 18, wherein the operations further comprise:
preventing exportation of the decrypted output data to unauthorized entities.

20. The non-transitory machine-readable storage medium of claim 18, wherein the operations further comprise:
embedding a digital watermark on or within the decrypted output data in association with the decrypting or the rendering.
